(19) United States
(12) Patent Application Publication
(10) Pub. No.: US 2026/0216394 A1
(43) Pub. Date: Jul. 30, 2026

(54) PROCESS CHALLENGE DEVICE AND STERILIZATION SYSTEM

(71) Applicant: Solventum Intellectual Properties Company, Maplewood, MN (US)

(72) Inventors: Francois Ahimou, Woodbury, MN (US); Timothy A. Kohman, Cottage Grove, MN (US); Naiyong Jing, St. Paul, MN (US)

(73) Assignee: Solventum Intellectual Properties Company, Maplewood, MN (US)

(21) Appl. No.: 19/144,980

(22) PCT Filed: Dec. 7, 2023
(86) PCT No.: PCT/IB2023/062369

§ 371 Date: Jul. 1, 2025

Related U.S. Application Data
(60) Provisional application No. 63/478,576, filed on Jan. 5, 2023.

(51) Int. Cl. A61L 2/28; A61L 2/07; A61L 103/15

(52) U.S. Cl. CPC A61L 2/28; A61L 2/07; A61L 2103/15; A61L 2202/121; A61L 2202/122

(57) ABSTRACT

A process challenge device to be used for determining the effectiveness of a sterilization procedure includes a pouch. The pouch includes an internal surface defining an internal storage space of the pouch, an external surface opposite to the internal surface, an inner film including polypropylene (PP) and an outer film including polyethylene terephthalate (PET). The outer film is disposed directly adjacent to and in contact with the inner film. The process challenge device further includes a test indicator received within the internal storage space of the pouch. The pouch has a total thickness equal to a sum of a thickness of the inner film and a thickness of the outer film. The total thickness being greater than or equal to 62 microns and less than or equal to 65 microns. The pouch has a water vapor transmission rate (WTVR) greater than 0.5 g/m²/24 hours and less than 3 g/m²/24 hours.

### TECHNICAL FIELD

The present disclosure relates generally to a process challenge device and a sterilization system, and more particularly, relates to the process challenge device to be used for determining the effectiveness of a sterilization procedure.

### BACKGROUND

Sterilization of medical and hospital equipment may not be effective until a steam sterilant has been in contact with all surfaces of materials being sterilized in a proper combination of time, temperature, and steam quality. In steam sterilizers, such as pre-vacuum steam sterilizers and gravity displacement steam sterilizers, the process of sterilization is conducted in three main phases. In the first phase, air is removed, including air trapped within any porous materials being processed. The first phase is therefore an air removal phase. The second phase is a sterilizing stage, in which a load (i.e., the articles being sterilized) is subjected to steam under pressure for a recognized, predetermined combination of time and temperature to effect sterilization. The third phase is a drying phase in which condensation formed during the first two phases is removed by evacuating the chamber.

Any air that is not removed from the sterilizer during the air removal phase of the cycle or which leaks into the sterilizer during a sub-atmospheric pressure stage due to, for example, faulty gaskets, valves, or seals, may form air pockets within any porous materials present. Such air pockets may create a barrier to steam penetration, thereby preventing adequate sterilizing conditions being achieved for all surfaces of the load during the sterilizing phase. For example, these air pockets may prevent the steam from reaching interior layers of materials, such as hospital linens or fabrics. In some other examples, these air pockets may prevent the steam from penetrating hollow spaces of tubes, catheters, syringe needles, and the like. Further, non-condensable gas (generally air) present within the sterilizer is a poor sterilant and may decrease sterilization efficacy. A percentage of non-condensable gas in the steam should be less than or equal to 3.5% by volume. Therefore, the presence of air pockets and/or non-condensable gas may affect a steam quality of the steam sterilant. As a result, proper sterilization may not occur due to reduced steam quality. A few more factors that may affect steam quality include insufficient steam supply, water quality, degassing, design of the sterilizer chamber, etc.

It can be stated that proper sterilization may not occur due to inappropriate steam quality, air removal, time, and temperature of sterilization. For monitoring whether the sterilization process is being conducted at adequate temperatures, with adequate steam quality, air removal, and for adequate time period, process challenge devices and/or Bowie-Dick test devices are used. Process challenge devices may be used to evaluate steam parameters, such as steam quality, temperature, and time of sterilization procedure. Bowie-Dick test devices are more focused on monitoring air removal inside the sterilizer chamber.

### SUMMARY

In a first aspect, the present disclosure provides a process challenge device to be used for determining the effectiveness of a sterilization procedure. The process challenge device includes a pouch. The pouch includes an internal surface defining an internal storage space of the pouch and an external surface opposite to the internal surface. The pouch further includes an inner film including at least 95% by weight of polypropylene (PP). The inner film includes the internal surface. The pouch further includes an outer film attached to the inner film and including at least 95% by weight of polyethylene terephthalate (PET). The outer film is disposed directly adjacent to and in contact with the inner film opposite to the internal surface. The outer film includes the external surface. The process challenge device includes a test indicator received within the internal storage space of the pouch. The pouch has a total thickness equal to a sum of a thickness of the inner film and a thickness of the outer film. The total thickness is greater than or equal to 62 microns and less than or equal to 65 microns. The pouch has a water vapor transmission rate (WTVR) greater than 0.5 g/m²/24 hours and less than 3 g/m²/24 hours.

In a second aspect, the present disclosure provides a sterilization system. The sterilization system includes a steam sterilizer including a sterilization chamber configured to receive a steam sterilant therein. The sterilization system includes a process challenge device removably received within the sterilization chamber and configured to be used for determining the effectiveness of a sterilization procedure. The process challenge device includes a pouch. The pouch includes an internal surface defining an internal storage space of the pouch and an external surface opposite to the internal surface. The pouch further includes an inner film including at least 95% by weight of polypropylene (PP). The inner film includes the internal surface. The pouch further includes an outer film attached to the inner film and including at least 95% by weight of polyethylene terephthalate (PET). The outer film is disposed directly adjacent to and in contact with the inner film opposite to the internal surface. The outer film includes the external surface. The process challenge device further includes a test indicator received within the internal storage space of the pouch. The pouch has a total thickness equal to a sum of a thickness of the inner film and a thickness of the outer film, the total thickness being greater than or equal to 62 microns and less than or equal to 65 microns. The pouch has a water vapor transmission rate (WTVR) greater than 0.5 g/m²/24 hours and less than 3 g/m²/24 hours.

### BRIEF DESCRIPTION OF THE DRAWINGS

Exemplary embodiments disclosed herein may be more completely understood in consideration of the following detailed description in connection with the following figures. The figures are not necessarily drawn to scale. Like numbers used in the figures refer to like components. However, it will be understood that the use of a number to refer to a component in a given figure is not intended to limit the component in another figure labeled with the same number.

FIG. 1 is a schematic block diagram of a sterilization system, according to an embodiment of the present disclosure;

FIG. 2 is a schematic top view of a process challenge device of the sterilization system, according to an embodiment of the present disclosure;

FIG. 3 is a sectional side view of the process challenge device taken along a line A-A′ as shown in FIG. 2, according to an embodiment of the present disclosure; and

FIG. 4 is an enlarged schematic view of a section X of a pouch of the process challenge device of FIG. 3, according to an embodiment of the present disclosure.

### DETAILED DESCRIPTION

In the following description, reference is made to the accompanying figures that form a part thereof and in which various embodiments are shown by way of illustration. It is to be understood that other embodiments are contemplated and may be made without departing from the scope or spirit of the present disclosure. The following detailed description, therefore, is not to be taken in a limiting sense.

As used herein, all numbers should be considered modified by the term “about”. As used herein, “a,” “an,” “the,” “at least one,” and “one or more” are used interchangeably.

The term “about”, unless otherwise specifically defined, means to a high degree of approximation (e.g., within +/−5% for quantifiable properties) but again without requiring absolute precision or a perfect match.

As used herein as a modifier to a property or attribute, the term “generally”, unless otherwise specifically defined, means that the property or attribute would be readily recognizable by a person of ordinary skill but without requiring absolute precision or a perfect match (e.g., within +/−20% for quantifiable properties).

The term “substantially”, unless otherwise specifically defined, means to a high degree of approximation (e.g., within +/−10% for quantifiable properties) but again without requiring absolute precision or a perfect match.

Terms such as same, equal, uniform, constant, strictly, and the like, are understood to be within the usual tolerances or measuring error applicable to the particular circumstance rather than requiring absolute precision or a perfect match.

As used herein, the terms “first” and “second” are used as identifiers. Therefore, such terms should not be construed as limiting of this disclosure. The terms “first” and “second” when used in conjunction with a feature or an element can be interchanged throughout the embodiments of this disclosure.

As used herein, “at least one of A and B” should be understood to mean “only A, only B, or both A and B”.

As used herein, the term “configured to” and like is at least as restrictive as the term “adapted to” and requires actual design intention to perform the specified function rather than mere physical capability of performing such a function.

As used herein, the terms “layer,” “sheet,”, “film”, or variations thereof, are used to describe an article having a thickness that is small relative to its length and width.

Steam sterilizers are widely used in medical centers and hospitals to sterilize medical equipment. Frequent testing or monitoring of steam quality may be essential to ensure a safe use of the medical equipment in a medical treatment. In other words, regular testing may have to be conducted to check effectiveness of air removal during air removal phase of the sterilization process, prior to subjecting the steam to a given load (i.e., medical equipment). One of the ways to monitor steam quality of the steam sterilant is by using a process challenge device.

Steam Flush Pressure Pulse (SFPP) sterilizers are one of the types of sterilizers that use a series of steam flushes and pressure pulses, in the preconditioning phase and prior exposure, to remove air from a sterilization chamber and/or medical equipment. The SFPP sterilizers are used in order to sterilize the medical equipment having lumened components, or surfaces that inhibit steam from contacting all surfaces. The SFPP sterilizer uses extended cycles with multiple pulses in the preconditioning phase in order to remove air from the surfaces that are hard to sterilize, such as lumens in the medical equipment.

Generally, the process challenge device containing the medical equipment to be sterilized along with an indicator is placed within the sterilization chamber of the SFPP sterilizer for monitoring an effective sterilization. The indicator monitors a defined resistance to steam during the sterilization process. The indicator may be used to assess the performance of a defined sterilization process. In order to effectively sterilize the medical equipment, the process challenge device should mimic a challenge/resistance presented by the medical equipment used in a sterilization process. The process challenge device provides a repeatable challenge to the sterilization process by representing worst-case conditions for the steam to be subjected within the sterilization chamber. While such process challenge devices have been useful, they have not always been convenient to use for extended cycles with longer exposure of steam.

The present disclosure relates to a process challenge device to be used for determining the effectiveness of a sterilization procedure. The process challenge device includes a pouch. The pouch includes an internal surface defining an internal storage space of the pouch and an external surface opposite to the internal surface. The pouch further includes an inner film including at least 95% by weight of polypropylene (PP). The inner film includes the internal surface. The pouch further includes an outer film attached to the inner film and including at least 95% by weight of polyethylene terephthalate (PET). The outer film is disposed directly adjacent to and in contact with the inner film opposite to the internal surface. The outer film includes the external surface. The process challenge device includes a test indicator received within the internal storage space of the pouch. The pouch has a total thickness equal to a sum of a thickness of the inner film and a thickness of the outer film. The total thickness being greater than or equal to 62 microns and less than or equal to 65 microns. The pouch has a water vapor transmission rate (WTVR) greater than 0.5 g/m²/24 hours and less than 3 g/m²/24 hours.

The process challenge device of the present disclosure is a two-film construction of the pouch having the inner film of polypropylene and the outer film of polyethylene terephthalate. The disclosed total thickness and the WTVR of the pouch may provide a required resistance to the steam sterilant and may therefore help in determining effectiveness of the sterilization procedure. In order to effectively sterilize a medical equipment, the process challenge device of the present disclosure may be able to mimic the challenge/resistance presented by the medical equipment used in a sterilization process. Hence, the inner film of polypropylene and the outer film of polyethylene terephthalate may represent worst-case conditions for the steam sterilant within the sterilization chamber.

In some embodiments, the process challenge device of the present disclosure may monitor extended cycles for steam flush pressure pulse (SFPP) sterilizers. It should be noted that the process challenge device may be useful for monitoring extended cycles with longer exposure of steam. Further, in comparison to conventional process challenge devices, the process challenge device of the present disclosure may provide a relatively greater resistance to the steam sterilant with a longer kill time enabling the test indicator to survive the long preconditioning phase before exposure to sterilization. Therefore, the process challenge device of the present disclosure may help in developing a more resistant biological or chemical indicator apparatus with longer exposure to the steam subjected inside the sterilization chamber.

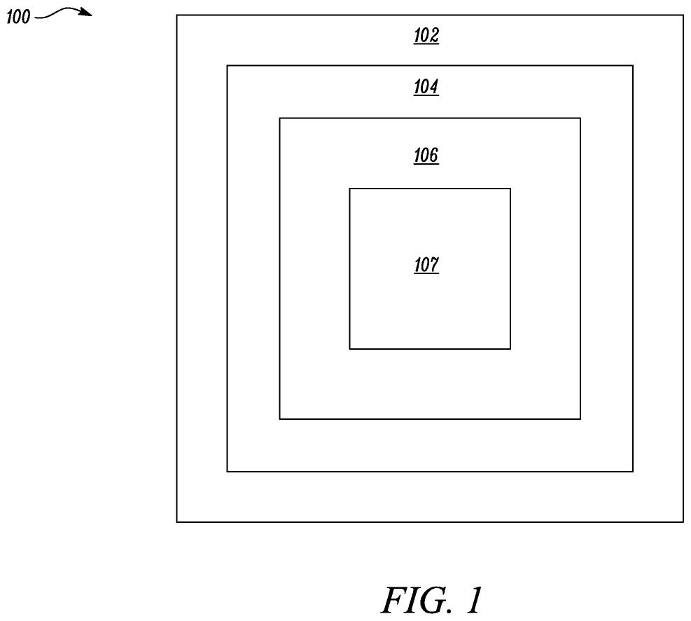

Referring now to Figures, FIG. 1 is a schematic block diagram of a sterilization system 100, according to an embodiment of the present disclosure. The sterilization system 100 includes a steam sterilizer 102 including a sterilization chamber 104 configured to receive a steam sterilant therein. The sterilization chamber 104 may have one or more environmental conditions. In some cases, the environmental condition may be related to conditions inside the sterilization chamber 104, and may include time, sterilant, temperature, pressure, or combinations thereof. In some embodiments, the sterilization chamber 104 may be made of various materials such as, but not limited to, steel, metal, polymer, or any other materials. When steam is used as the steam sterilant, an object of a sterilization process is to bring steam at an appropriate temperature into contact with all surfaces of the articles being sterilized for an appropriate period of time.

The sterilization system 100 includes a process challenge device 106 removably received within the sterilization chamber 104 and configured to be used for determining the effectiveness of a sterilization procedure. The process challenge device 106 may be used to conduct Bowie-Dick tests and offer a resistance to the steam sterilant. In some embodiments, the process challenge device 106 may be adapted to be used to determine effectiveness of the sterilization procedure selected from the group consisting of 121° C. gravity process, 121° C. pre-vac process, 121° C. SFPP process, 132° C. gravity process, 132° C. pre-vac process, 132° C. SFPP process, 134° C. pre-vac process, 134° C. SFPP process, 135° C. gravity process, 135° C. pre-vac process, and 135° C. SFPP process. SFPP means steam flush pressure pulse sterilization cycle and pre-vac means pre-vacuum or vacuum-assisted sterilization cycle.

The process challenge device 106 includes a test indicator 107. In some embodiments, the test indicator 107 may be a biological indicator (BI) or a chemical indicator (CI). The biological indicator may include viable microorganisms with a defined resistance to the sterilization process and may indicate the effectiveness of the sterilization procedure. The chemical indicator may include one or more chemicals that may visibly change their color to indicate the effectiveness of the sterilization procedure. The test indicator 107 may be chosen to be used with sterilization conditions to be employed in a particular sterilization process. Moreover, the test indicator 107 may be chosen based upon an amount of exposure to sterilization conditions required to cause the test indicator 107 to indicate that the exposure has occurred. The choice of the test indicator 107 may thereby be used to increase or decrease the resistance of the process challenge device 106.

In some other embodiments, the process challenge device 106 may include, but not limited to, any type of consumer or industrial item, medical product, pharmaceutical item, or food item to sterilize within the sterilization chamber 104.

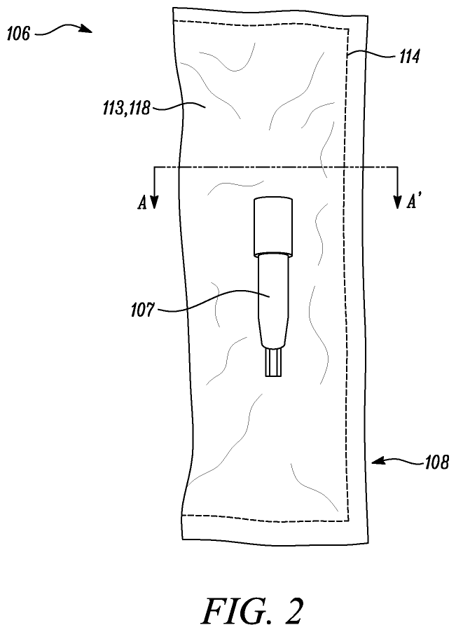

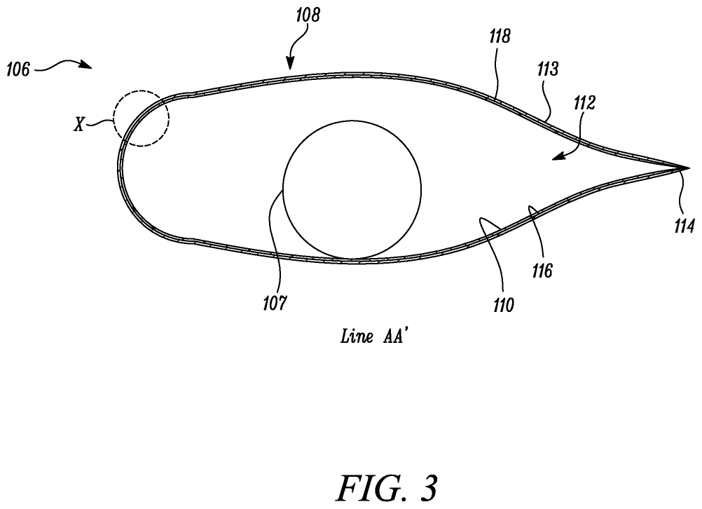

FIG. 2 is a schematic top view of the process challenge device 106, according to an embodiment of the present disclosure. The process challenge device 106 includes a pouch 108. FIG. 3 is a sectional side view of the process challenge device 106 taken along a line A-A′ as shown in FIG. 2, according to an embodiment of the present disclosure.

With reference to FIGS. 2 and 3, the pouch 108 includes an internal surface 110 defining an internal storage space 112 of the pouch 108. The test indicator 107 is received within the internal storage space 112 of the pouch 108. In some embodiments, there may be two or more test indicators 107 received within the internal storage space 112 of the pouch 108. The pouch 108 further includes an external surface 113 opposite to the internal surface 110. In some embodiments, the pouch 108 is devoid of any through holes extending from the internal surface 110 to the external surface 113.

In some embodiments, the pouch 108 further includes a peripheral seal 114, such that the internal storage space 112 of the pouch 108 is sealed. In some embodiments, the peripheral seal 114 is a heat seal. In some embodiments, the peripheral seal 114 may be an ultrasonic seal. In some other embodiments, the internal storage space 112 of the pouch 108 may be sealed by an adhesive, including a pressure sensitive adhesive. In some cases, the pouch 108 may include a flexible sheet that may be folded in such a way that one or more side walls of the flexible sheet may overlap, thereby creating the internal storage space 112 of the pouch 108. The peripheral seal 114 may couple the overlapping side walls of the pouch 108.

The pouch 108 further includes an inner film 116 including at least 95% by weight of polypropylene (PP). The inner film 116 includes the internal surface 110. In some embodiments, the inner film 116 includes at least 99% by weight of polypropylene. In some examples, the polypropylene may be a cast polypropylene (CPP), an oriented polypropylene (OPP), or any combination thereof. In some other examples, the inner film 116 may also include nylon.

The pouch 108 further includes an outer film 118 attached to the inner film 116 and including at least 95% by weight of polyethylene terephthalate (PET). In some embodiments, the outer film 118 includes at least 99% by weight of polyethylene terephthalate. Polyethylene terephthalate is a polyester that refers to a homopolymer or a copolymer having an ester linkage between monomer units, or a homopolymer or a copolymer of alkyl-aromatic esters, including but not limited to amorphous polyethylene terephthalate (APET), polyethylene furanoate (PEF), glycol-modified polyethylene terephthalate (PETG), and polybutylene terephthalate (PBT); or a copolymer of terephthalate and isophthalate including but not limited to polyethylene terephthalate/isophthalate copolymer, such as isophthalic acid (IPA) (modified polyethylene terephthalate (PETI). The outer film 118 is disposed directly adjacent to and in contact with the inner film 116 opposite to the internal surface 110. Specifically, the outer film 118 includes the external surface 113.

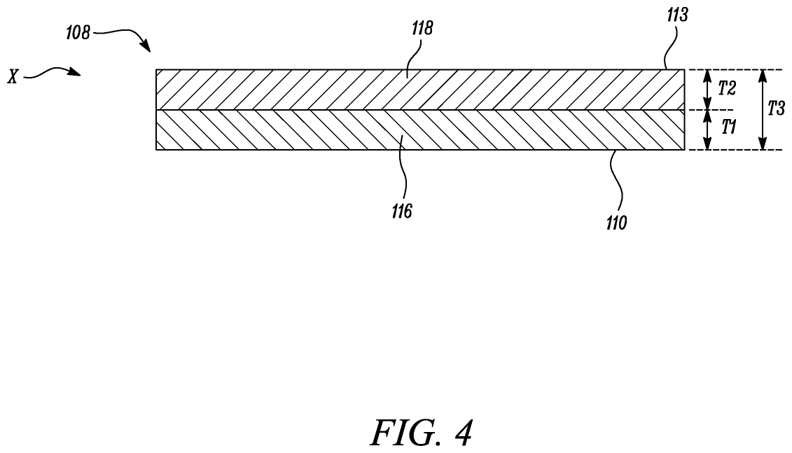

FIG. 4 is an enlarged schematic view of a section X (shown in FIG. 3) of the pouch 108, according to an embodiment of the present disclosure. With reference to FIGS. 2 to 4, the pouch 108 has a total thickness T3 equal to a sum of a thickness T1 of the inner film 116 and a thickness T2 of the outer film 118. In some embodiments, the thickness T1 of the inner film 116 is equal to the thickness T2 of the outer film 118. In some other embodiments, the thickness T1 of the inner film 116 is different from the thickness T2 of the outer film 118. The total thickness T3 is greater than or equal to 62 microns and less than or equal to 65 microns.

With reference to FIGS. 1 to 4, the pouch 108 has a water vapor transmission rate (WTVR) or moisture vapor transmission rate (MVTR) greater than 0.5 g/m²/24 hours and less than 3 g/m²/24 hours. In some embodiments, the WVTR/MVTR of the pouch 108 is 0.96 g/m²/24 hours. MVTR is a mass of steam sterilant (in grams) that passes through a given area of the pouch 108 (in square meters) at a specified temperature and humidity over a given period of time. In other words, MVTR is an amount of steam sterilant that passes through the pouch 108, when the steam is subjected inside the sterilization chamber 104 during the sterilization process. WVTR may be hereinafter interchangeably referred to as MVTR.

The process challenge device 106 forms a two-film construction in the form of the pouch 108 including the inner film 116 of polypropylene and the outer film 118 of polyethylene terephthalate. The disclosed total thickness T3 and the WTVR of the pouch 108 may provide a required resistance to the steam sterilant and may therefore help in determining effectiveness of the sterilization procedure. In order to effectively sterilize a medical equipment, the process challenge device 106 may be able to mimic the challenge/resistance presented by the medical equipment used in the sterilization process. Hence, the inner film 116 of polypropylene and the outer film 118 of polyethylene terephthalate may represent worst-case conditions for the steam sterilant within the sterilization chamber 104.

In some embodiments, the process challenge device 106 may monitor extended cycles for steam flush pressure pulse (SFPP) sterilizers. It should be noted that the process challenge device 106 may be useful for monitoring extended cycles with longer exposure of steam. Further, in comparison to conventional process challenge devices, the process challenge device 106 of the present disclosure may provide a relatively greater resistance to the steam sterilant with a longer kill time enabling the test indicator 107 to survive the long preconditioning phase before exposure to sterilization. Therefore, the process challenge device 106 may help in developing a more resistant biological or chemical indicator apparatus with longer exposure to the steam subjected inside the sterilization chamber 104.

### MVTR Measurement Method

MVTR may be measured using a modified Payne cup method. A 1.5 inches diameter sample was cut by a circular die cutter ensuring that the sample was large enough to completely cover elliptical holes of two adhesive foil rings. Liners were removed from the two adhesive foil rings and the sample was placed between the adhesive-containing surfaces of the two foil adhesive rings. The elliptical holes of each adhesive foil ring were carefully aligned with each other. Finger pressure was used to form a foil assembly (i.e., a sample assembly placed between the adhesive foil rings) that was flat, wrinkle free, and had no void areas in the exposed sample area.

A 4 oz glass bottle was filled with approximately 50 mL of water using a plastic beaker. A rubber washer was placed on a lip of the bottle and the foil assembly was placed on the rubber washer at the center of the bottle. A lid of the bottle was then screwed loosely on the bottle.

The bottle was placed upright into a wire basket. The wire basket along with the bottle was placed in the sterilization chamber 104 at 40 degrees Celsius and 20% relative humidity for 4-6 hours. At the end of 6 hours (conditioning of the bottle), the lid of the bottle was tightened inside the sterilization chamber 104 so that the sample was level with the lid (no bulging) and the rubber washer was in proper seating position.

The wire basket was removed from the sterilization chamber 104 and the bottle was weighed immediately to the nearest 0.01 gram for an initial weight W₁ (refer to Equation 1). The bottle was then placed upright into the wire basket and the wire basket was returned to the sterilization chamber 104 for 18-24 hours. Date and time (in hours) were recorded when the bottle was put into the sterilization chamber 104. The basket was removed after time h1, and the bottle was weighed immediately to the nearest 0.01 g for a final weight W₂. Date and time h2 (in hours) were recorded when the bottle was removed from the sterilization chamber 104.

The Moisture Vapor Transmission Rate (MVTR) in g/m²/24 hours of the sample is calculated according to Equation 1 provided below:

$$M V T R ( g / {m}^{2} / 24 hours ) = ( ( {W}_{1} - {W}_{2} ) * 24 ) / ( A * T ) \qquad ( Equation 1 )$$

where, W₁ is an initial weight of the bottle in grams;
W₂ is a final weight of the bottle in grams; 24 is normalization of MVTR to 24 hours;
A is an exposed area of the sample in square meter; and
T is time spent by the sample inside the sterilization chamber 104 in hours i.e., h2−h1.

### TESTS

### Test 1

A test was performed to determine MVTR of three different pouches with different thicknesses. Three different pouches included the pouch 108 (shown in FIG. 2), a pouch A and a pouch B. Particularly, the pouch 108 was constructed of PP and PET, and had a thickness of 62-65 microns. The pouch A was constructed of metallized PET and had a thickness of 84-89 microns. The pouch B was constructed of foil (made of a metal) and had a thickness of 108-112 microns. Further, each of the pouch 108, the pouch A, and the pouch B carried 1.5 kgs of surgical instruments.

In this test, each of the pouch 108, the pouch A, and the pouch B was placed inside the sterilization chamber 104 (shown in FIG. 1) and was subjected to the sterilization process. The temperature inside the sterilization chamber 104 of the steam sterilizer 102 was maintained at around 132 degrees Celsius.

The average MVTR was measured in gram per square meter per 24 hours (g/m²/24 hours) for each of the pouch 108, the pouch A, and the pouch B. The measured values of the average MVTR are summarized in Table 1 provided below.

TABLE 1

| | Pouch 108 | Pouch A | Pouch B |
|---|---|---|---|
| MVTR | 0.96 g/m²/ | 0.012 g/m²/ | 0.0004 g/m²/ |
| | 24 hours | 24 hours | 24 hours |
| Thickness | 62-65 | 84-89 | 108-112 |
| | microns | microns | microns |

The data of Table 1 demonstrates that when the pouch 108 was sterilized inside the sterilization chamber 104, the average MVTR recorded was 0.96 g/m²/24 hours. Further, when the pouch A was sterilized inside the sterilization chamber 104, the average MVTR recorded was 0.012 g/m²/24 hours. The data demonstrates that the average MVTR of the pouch A deteriorates when the thickness of the pouch A was increased (relative to the thickness of the pouch 108). Specifically, the average MVTR of the pouch 108 was almost 8 times more effective than the pouch A. Moreover, when the pouch B was sterilized inside the sterilization chamber 104, the average MVTR recorded was 0.0004 g/m²/24 hours. Therefore, the thickness of the pouch A and pouch B negatively impacted the average MVTR of the respective pouch. Since the average MVTR was highest for the pouch 108, the surgical instruments loaded inside the pouch 108 were properly sterilized. As a result, the pouch 108 of the process challenge device 106 may precisely determine the effectiveness of the sterilization procedure.

### Test 2

A test was performed to determine the performance of biological indicators received inside three different pouches. Three different pouches included the pouch 108 (shown in FIG. 2), the pouch A and the pouch B. Further, each of the pouch 108, the pouch A, and the pouch B carried 1.5 kgs of surgical instruments. Moreover, during the test, the test indicator 107 could be the biological indicator received inside each of the pouch 108, the pouch A, and the pouch B.

In this test, each of the pouch 108, the pouch A, and the pouch B was placed inside the sterilization chamber 104 (shown in FIG. 1) and was subjected to the sterilization process. The temperature inside the sterilization chamber 104 of the steam sterilizer 102 was maintained at around 132 degrees Celsius. Further, the biological indicator received inside each of the pouch 108, the pouch A, and the pouch B was incubated for 0 minute and 6 minutes. For the biological indicator in each of the pouch 108, the pouch A, and the pouch B, the test was performed to identify fluorescence indicative of complete inactivation of the test microorganisms or survival/growth of at least a portion of the test microorganisms after exposure to the sterilization process.

The performance of each biological indicator received inside corresponding pouch at 0 minute & 6 minutes are summarized in Table 2 provided below.

TABLE 2

| | Pouch 108 | | Pouch A | | Pouch B | |
|---|---|---|---|---|---|---|
| Exposure time | 0 | 6 | 0 | 6 | 0 | 6 |
| (in minutes) | | | | | | |
| Fluorescence | Pass | Pass | Pass | Fail | Pass | Fail |
| Growth | Pass | Pass | Pass | Fail | Pass | Fail |

The data of Table 2 demonstrates that the pouch 108 passed a biological indicator survival at 0 minute exposure and a full kill after 6 minute exposure time to the steam sterilization cycle. The full kill implies exposure time (i.e., 6 minutes) to achieve inactivation of a population of viable microorganisms of the biological indicator. The pouch A passed the biological indicator survival at 0 minute exposure but failed the full kill after 6 minute exposure time to the steam sterilization cycle. The pouch B passed the biological indicator survival at 0 minute exposure and failed the full kill after 6-minute exposure time to the steam sterilization cycle.

Unless otherwise indicated, all numbers expressing feature sizes, amounts, and physical properties used in the specification and claims are to be understood as being modified by the term “about”. Accordingly, unless indicated to the contrary, the numerical parameters set forth in the foregoing specification and attached claims are approximations that can vary depending upon the desired properties sought to be obtained by those skilled in the art utilizing the teachings disclosed herein.

Although specific embodiments have been illustrated and described herein, it will be appreciated by those of ordinary skill in the art that a variety of alternate and/or equivalent implementations can be substituted for the specific embodiments shown and described without departing from the scope of the present disclosure. This application is intended to cover any adaptations or variations of the specific embodiments discussed herein. Therefore, it is intended that this disclosure be limited only by the claims and the equivalents thereof.

1. A process challenge device to be used for determining the effectiveness of a sterilization procedure, the process challenge device comprising:
a pouch comprising:
an internal surface defining an internal storage space of the pouch;
an external surface opposite to the internal surface;
an inner film comprising at least 95% by weight of polypropylene (PP), the inner film comprising the internal surface; and
an outer film attached to the inner film and comprising at least 95% by weight of polyethylene terephthalate (PET), wherein the outer film is disposed directly adjacent to and in contact with the inner film opposite to the internal surface, the outer film comprising the external surface; and
a test indicator received within the internal storage space of the pouch;
wherein:
the pouch has a total thickness equal to a sum of a thickness of the inner film and a thickness of the outer film, the total thickness being greater than or equal to 62 microns and less than or equal to 65 microns; and
the pouch has a water vapor transmission rate (WTVR) greater than 0.5 g/m²/24 hours and less than 3 g/m²/24 hours.

2. The process challenge device of claim 1, wherein the pouch is devoid of any through holes extending from the internal surface to the external surface.

3. The process challenge device of claim 1, wherein the pouch further comprises a peripheral seal, such that the internal storage space of the pouch is sealed.

4. The process challenge device of claim 3, wherein the peripheral seal is a heat seal.

5. The process challenge device of claim 1, wherein the WVTR of the pouch is 0.96 g/m²/24 hours.

6. The process challenge device of claim 1, wherein the test indicator is a biological indicator or a chemical indicator.

7. The process challenge device of claim 1, wherein the inner film comprises at least 99% by weight of polypropylene.

8. The process challenge device of claim 1, wherein the outer film comprises at least 99% by weight of polyethylene terephthalate.

9. The process challenge device of claim 1, wherein the thickness of the inner film is equal to the thickness of the outer film.

10. The process challenge device of claim 1, wherein the thickness of the inner film is different from the thickness of the outer film.

11. A sterilization system comprising:
a steam sterilizer comprising a sterilization chamber configured to receive a steam sterilant therein; and
a process challenge device removably received within the sterilization chamber and configured to be used for determining the effectiveness of a sterilization procedure, the process challenge device comprising:
a pouch comprising:
an internal surface defining an internal storage space of the pouch;
an external surface opposite to the internal surface;
an inner film comprising at least 95% by weight of polypropylene (PP), the inner film comprising the internal surface; and
an outer film attached to the inner film and comprising at least 95% by weight of polyethylene terephthalate (PET), wherein the outer film is disposed directly adjacent to and in contact with the inner film opposite to the internal surface, the outer film comprising the external surface; and
a test indicator received within the internal storage space of the pouch;
wherein:
the pouch has a total thickness equal to a sum of a thickness of the inner film and a thickness of the outer film, the total thickness being greater than or equal to 62 microns and less than or equal to 65 microns; and
the pouch has a water vapor transmission rate (WTVR) greater than 0.5 g/m²/24 hours and less than 3 g/m²/24 hours.

12. The sterilization system of claim 11, wherein the pouch is devoid of any through holes extending from the internal surface to the external surface.

13. The sterilization system of claim 11, wherein the pouch further comprises a peripheral seal, such that the internal storage space of the pouch is sealed.

14. The sterilization system of claim 13, wherein the peripheral seal is a heat seal.

15. The sterilization system of claim 11, wherein the WVTR of the pouch is 0.96 g/m²/24 hours.

16. The sterilization system of claim 11, wherein the test indicator is a biological indicator or a chemical indicator.

17. The sterilization system of claim 11, wherein the inner film comprises at least 99% by weight of polypropylene.

18. The sterilization system of claim 11, wherein the outer film comprises at least 99% by weight of polyethylene terephthalate.

19. The sterilization system of claim 11, wherein the thickness of the inner film is equal to the thickness of the outer film.

20. The sterilization system of claim 11, wherein the thickness of the inner film is different from the thickness of the outer film.
